(19) United States
(12) Patent Application Publication
(10) Pub. No.: US 2025/0090641 A1
(43) Pub. Date: Mar. 20, 2025

(54) EXPRESSION VECTOR FOR CHOLESTEROL 24-HYDROLASE IN THERAPY OF POLYGLUTAMINE REPEAT SPINOCEREBELLAR ATAXIAS

(71) Applicants: BRAINVECTIS, Paris (FR); INSERM (INSTITUT NATIONAL DE LA SANTÉ ET DE LA RECHERCHE MÉDICALE), Paris (FR); UNIVERSIDADE DE COIMBRA, Coimbra (PT); CENTRO DE NEUROCIÊNCIAS E BIOLOGIA CELULAR, Coimbra (PT)

(72) Inventors: Sandro ALVES, Paris (FR); Nathalie CARTIER-LACAVE, Paris (FR); Luis PEREIRA DE ALMEIDA, Coimbra (PT); Clévio NOBREGA, Coimbra (PT); Liliana MENDONÇA, Coimbra (PT)

(21) Appl. No.: 18/905,742

(22) Filed: Oct. 3, 2024

Related U.S. Application Data
(62) Division of application No. 16/480,541, filed on Jul. 24, 2019, now Pat. No. 12,138,298.

(30) Foreign Application Priority Data
Jan. 30, 2017 (EP) 17305100.4

(51) Int. Cl. A61K 38/44; A61K 48/00; A61P 25/28; C12N 7/00; C12N 15/86

(52) U.S. Cl. CPC A61K 38/44; A61K 48/0075; A61P 25/28; C12N 7/00; C12N 15/86; C12Y 114/13098; A61K 48/00; C12N 2750/14143; C12N 2750/14171

(57) ABSTRACT

The present invention relates to a vector for use in the treatment of a polyglutamine repeat spinocerebellar ataxia, which vector comprises cholesterol 24-hydroxylase encoding nucleic acid.

### RELATED APPLICATIONS

This application is a Divisional of U.S. application Ser. No. 16/480,541 filed Jul. 24, 2019, which is a U.S. National Stage Entry of PCT/EP2018/052199 filed Jan. 30, 2018, which claims priority to European Patent Application No. 17305100 filed Jan. 30, 2017.

The present invention relates to treatment of polyglutamine repeat spinocerebellar ataxias.

### BACKGROUND OF THE INVENTION

Spinocerebellar ataxias (SCA) belong to the group of neurodegenerative disorders characterized by slowly progressive in-coordination of gait and troubles of balance, often associated with poor coordination of hands, speech and eye movements. Spinocerebellar ataxias result in unsteady and clumsy motion of the body due to a failure of the fine coordination of muscle movements.

Spinocerebellar ataxias are associated with progressive disappearance of part of the nervous system cells located in the cerebellum, leading to atrophy of the cerebellum. Spinocerebellar ataxias are genetic disorders associated with different gene alterations. Numerous types of spinocerebellar ataxias are now known, for which specific genetic information is available. The first ataxia gene was identified in 1993 for a dominantly inherited type, and was called “Spinocerebellar ataxia type 1” (SCA1). Subsequently, as additional dominant genes were found, they were called SCA2, SCA3, etc. Usually, the “type number of SCA refers to the order in which the gene was found. At this time, there are at least 29 different gene mutations which have been found.

The most common alteration consists on an excessive repetition of the trinucleotide cytosine-adenine-guanine (CAG) in some genes located in different chromosomes. Those ataxias with poly CAG expansions are referred to as polyglutamine repeat spinocerebellar ataxias. The excessive repetition of CAG in a gene may lead to the repetition of glutamine in the protein encoded by said gene, producing abnormal protein.

As an example, Spinocerebellar ataxia type 3 (SCA3) also called Machado-Joseph disease (MJD) is a progressive autosomal dominant inherited adult-onset neurodegenerative disorder (Durr et al., 1996), caused by a CAG-repeat expansion in the coding region of the MJD1 gene, which results in an abnormally expanded polyglutamine (PolyQ) tract in the coding region of the ataxin-3 (ATXN3) gene (Kawaguchi et al., 1994). It confers a toxic gain of function to the expanded ATXN3 protein which abnormally accumulates in neuronal cells, thus promoting the formation of insoluble intranuclear inclusions (Orr and Zoghbi, 2007; Schols et al., 2004). SCA3 is clinically revealed as a range of progressive cerebellar dysfunctions unpredictably associated with degeneration of the striatum, substantia nigra and cranial nerve motor nuclei (Alves et al., 2008b; Durr et al., 1996; Klockgether et al., 1998; Rub et al., 2008), therefore resulting in a broad spectrum of symptoms such as ataxia, lack of motor coordination, postural instability, parkinsonism, pyramidal signs and neuropathy among other symptoms (Burk et al., 1999; Rub et al., 2006; Schols et al., 1997; Schols et al., 2004). A spinal cord involvement has also been described (Lukas et al., 2008).

The general prevalence of SCA is of 1 for 125,000, but in some part of the world, such as in the Azorean islands, the prevalence of SCA3 is up to 1 for 140.

Up to now, there is no known cure for SCA. Treatments are generally limited to softening symptoms. There is thus an acute need to develop new strategies for therapy in SCA in general and polyglutamine repeat spinocerebellar ataxias (PolyQ SCA) in particular.

### SUMMARY OF THE INVENTION

The inventors have shown a dysfunction of the cholesterol metabolism in patients with a PolyQ SCA. Based on this discovery, the inventors successfully studied the possibility to address the modulation of the cholesterol metabolism pathway as a therapeutic strategy to counteract PolyQ SCA, by use of a vector comprising cholesterol 24-hydroxylase encoding nucleic acid that expresses cholesterol 24-hydroxylase in the target cells.

It is therefore an object of the present invention to provide a vector for use in the treatment of a polyglutamine repeat spinocerebellar ataxia, which vector comprises cholesterol 24-hydroxylase encoding nucleic acid.

In an embodiment, the Polyglutamine repeat spinocerebellar ataxia selected from the group of Spinocerebellar ataxia type 1 (SCA1), Spinocerebellar ataxia type 2 (SCA2), Spinocerebellar ataxia type 3 (SCA3), Spinocerebellar ataxia type 6 (SCA6), Spinocerebellar ataxia type 7 (SCA7) and Spinocerebellar ataxia type 17 (SCA17).

In an embodiment, the vector comprises a nucleic acid sequence that encodes the amino acid sequence SEQ ID No. 2. Alternatively, the vector comprises the nucleic acid sequence SEQ ID No. 1.

In an embodiment, the vector is selected from the group of adenovirus, lentivirus, retrovirus, herpes-virus and Adeno-Associated Virus (AAV) vectors, preferably an AAV vector, more preferably an AAV9 or AAV10 (AAVrh.10) vector, even more preferably an AAVrh.10.

In an embodiment, the vector is to be administered directly into the brain of the patient, preferably to cerebellum, striatum, brainstem, frontotemporal lobes and/or visual cortex, more preferably to cerebellum. Alternatively or in addition, the vector is to be administered into the spinal cord of the patient.

It is another object of the invention to provide a pharmaceutical composition for use in the treatment of polyglutamine repeat spinocerebellar ataxias, which comprises a vector comprising cholesterol 24-hydroxylase encoding nucleic acid.

### FIGURES

The patent or application file contains at least one drawing executed in color. Copies of this patent or patent application publication with color drawing(s) will be provided by the Office upon request and payment of the necessary fee.

FIG. 1—Representative western-blot showing the levels of the HA tag in the cerebellum and “rest of the brain” of MJD transgenic mice (n=6) vs. wild-type controls (n=6). Frozen biopsies from six MJD transgenic mice expressing the N-terminal truncated human ATXN3, with 69 CAG repeats and six age-matched control littermates were used;

FIG. 2—Representative western-blots showing the levels of CYP46A1 protein in the brain of MJD mice (n=6) and wild-type controls (n=6). Optical densitometry revealing 24.3% reduction in CYP46A1 protein levels (Mann-Whitney test; p=0.026) in cerebellar biopsies from MJD mice relatively to control littermates (A and B). No statistical significant differences were found in biopsies from the “rest of the brain” in MJD mice and controls (Mann-Whitney test; p=0.13) (C and D);

FIG. 3—Quantitative measurements of both sterols and oxysterols by gas-chromatography mass spectrometry (GC/MS) in cerebellar extracts and also biopsies from the “rest of the brain” from MJD Q69 mice (n=6) and age-matched littermates (n=6), showing impairments in the cholesterol metabolism pathway in MJD mice relatively to control littermates;

FIG. 4—AAV-based CYP46A1 overexpression alleviates neuropathology in an LV-based mouse model of MJD, 2 months post-injection. A) Representative immunohistochemistry detecting the presence of the HA tag in AAVrh.10-CYP46A1-injected LV-MJD mice but not in the control group injected with AAVrh.10-GFP; in mice injected with AAVrh.10-GFP, widespread GFP immunoreactivity is detected in the mouse striatum whereas no HA signal is detected. Immunohistochemistry and quantitative analysis for mutant ataxin-3-positive inclusions (revealed with the 1H9 antibody) (A) revealed a statistically significant decrease (˜59%) in the number of ataxin-3-positive inclusions (2751±267.8; n=8) compared to the control group (GFP-co-injected mice; 6684±717.2 inclusions; n=8; Student's T test, P=0.0002) (B). A statistically significant decrease in the size of inclusions (˜47%) in AAVrh.10-CYP46A1 treated LV-MJD mice (59.29±4.67 arbitrary units (a.u.); n=8) compared to LV-MJD mice injected with the control AAVrh.10-GFP (111.4±3.96 a.u.; n=8; Student's T test, P<0.0001) was observed (C). Anti-ubiquitin staining that demonstrated a reduction in the number of ubiquitinated inclusions (qualitative analysis) in LV-MJD mice injected with AAVrh.10-CYP46A1 relatively to LV-MJD mice injected with control AAVrh.10-GFP (A). CYP46A1 overexpression led to a statistically significant reduction (˜48%) of DARPP-32 depleted region (0.48±0.77 mm³; n=8) relatively to LV-MJD mice injected with control AAVrh.10-GFP (0.91±0.14 mm³; n=8) Student's T test; P=0.015).

FIG. 5: Effect of AAV-CYP46A1 expression in the motor coordination impairments of MJD-transgenic mice. Non injected wild-type mice (WT) and MJD-transgenic mice injected with AAV10 encoding for CYP46A1 (CYP) or GFP (GFP) were assessed for motor coordination performance over 10 weeks. Mice were tested for A) rotarod at constant velocity, B) accelerated rotarod and C) hind base test (footprinting test). A significant improvement of the motor performance in the transgenic MJD mice treated with CYP46A1 as compared to the control mice (GFP) was observed. Data are presented as mean±SEM. Two-way ANOVA analysis with Bonferroni post-test; *p<0.05. CYP: n=10; GFP: n=9; WT: n=4.

FIG. 6: AAV-CYP46A1 overexpression decreases aggregation and mitigates Purkinje cell loss in the cerebellum of MJD-transgenic mice. Immunohistochemistry in MJD-transgenic mice injected with AAV10 encoding for CYP46A1 (CYP) or GFP (GFP) demonstrates a decrease of ubiquitinated aggregates (A). AAV10-CYP46A1 injected MJD transgenic mice display increased average number of Purkinje cells (B) and increased cerebellar volume (C) relatively to MJD transgenic mice treated with the control vector (AAV10-GFP).

FIG. 7: Representative western-blot depicting the levels of mutant ATXN3 upon CYP46A1 overexpression in Neuro2A cells (A). Optical densitometry analysis revealed a 66% and 90% reduction of mutant ATXN3 aggregates (B) and soluble levels (C), respectively, upon CYP46A1 overexpression (Student t-test, p=0.0033; n=4).

FIG. 8: Representative western-blot depicting the levels of autophagy marker LC3B-II upon CYP46A1 overexpression in Neuro2A cells (A). Optical densitometry analysis revealed an increase in autophagy upon CYP46A1 overexpression highlighted by an increase in the LC3B-II compared to controls conditions, both with and without autophagy inhibitor chloroquine (B). Moreover, CYP46A1 overexpression induced an increase in LC3B-II net flux compared to control levels, suggesting an activation of autophagic flux (C). (*P<0.05, **P<0.01; ***P<0.001; ****P<0.0001; Unpaired Student's t-test; n=4).

### DETAILED DESCRIPTION OF THE INVENTION

The inventors demonstrated that delivering a vector expressing a CYP46A1 gene into the brain of a mouse model of Polyglutamine repeat spinocerebellar ataxia, resulted in a significant decrease of neuronal dysfunction, and more particularly of cerebellum attacks.

On this basis, the inventors provide a viral vector for the treatment of Polyglutamine repeat spinocerebellar ataxias, wherein the vector expresses CYP46A1 in cells of the central nervous system.

### Polyglutamine Repeat Spinocerebellar Ataxias

The present invention specifically relates to treatment of polyglutamine repeat spinocerebellar ataxias, which are caused when a disease-associated protein (i.e., ataxin-1, ataxin-3, etc.) contains an abnormal number of repeats of glutamine residues, termed a polyQ or a “CAG trinucleotide repeat” disease.

In the context of the invention, the terms “treatment”, “treat” or “treating” are used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

As used herein, the term “subject” or “patient” refers to an animal, preferably to a mammal, even more preferably to a human, including adult and child. However, the term “subject” can also refer to non-human animals, in particular mammals such as mouse, and non-human primates.

The present invention relates preferably to a Polyglutamine repeat spinocerebellar ataxia selected from the group of Spinocerebellar ataxia type 1 (SCA1), Spinocerebellar ataxia type 2 (SCA2), Spinocerebellar ataxia type 3 (SCA3), Spinocerebellar ataxia type 6 (SCA6), Spinocerebellar ataxia type 7 (SCA7) and Spinocerebellar ataxia type 17 (SCA17).

SCA1, or SCA type 1, is characterized by abnormal CAG repeats in the gene encoding Ataxin 1 protein (ATXN1). This elongation is variable in length. The shortest repeat length shown to cause SCA1 is 39 uninterrupted CAG triplets, and the longest observed to date contained 83 repeats. Longer repeat tracts are correlated with earlier age of onset and faster progression. Mutant ATXN1 protein spontaneously misfolds and forms aggregates in the nucleus of Purkinje neurons.

SCA2, or SCA type 2, is characterized by abnormal CAG repeats in the coding region of the ATXN2 gene producing an elongated polyglutamine tract in the corresponding protein. The expanded repeats are variable in size and unstable, usually increasing in size when transmitted to successive generation. The locus has been mapped to chromosome 12, and it has been determined that the disease allele usually contains 34 to 52 CAG repeats, but can contain as few as 32 or more than 100.

SCA3, or SCA type 3, also called Machado-Joseph disease (MJD), is characterized by abnormal CAG repeats in the gene encoding the Ataxin 3 protein (ATXN3). The expansion of these repeats from the normal 13-36 to the abnormal 68-79 is the cause of MJD. There is an inverse correlation between the age of onset and CAG repeat numbers.

SCA6, or SCA type 6, is characterized by an insertion that extends the coding region of CACNA1A gene, that encodes the Caᵥ2.1 P/Q voltage-dependent calcium channel, to include from 4-16 to 21-28 CAG repeats.

SCA7, or SCA type 7, is characterized by abnormal CAG repeats in the coding region of the ATXN7 gene, producing an elongated polyglutamine tract in the corresponding protein (ATXN7).

SCA17, or SCA type 17, is characterized by abnormal CAG repetitions in the coding region of the gene encoding the TATA-binding protein (TBP). TBP is characterized by a long string of glutamines in the N-terminus of the protein. This region modulates the DNA binding activity of the C-terminus, and modulation of DNA-binding affects the rate of transcription complex formation and initiation of transcription. The expand of the number of CAG repeats thus increases the length of the glutamine string and thereby affect the DNA binding activity of the TBP.

### The CYP46A1 Sequences

A first object of the invention relates to a vector for use in the treatment of a PolyQ SCA, which comprises the full sequence of cholesterol 24-hydroxylase encoding nucleic acid.

As used herein, the term “gene” refers to a polynucleotide containing at least one open reading frame that is capable of encoding a particular polypeptide or protein after being transcribed or translated.

As used herein, the terms “coding sequence” or “a sequence which encodes a particular protein”, denotes a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

The CYP46A1 gene encodes cholesterol 24-hydroxylase. This enzyme is a member of the cytochrome P450 superfamily of enzymes. The enzyme converts cholesterol into 24S-hydroxycholesterol (24S-OH-Chol) that can dynamically cross the BBB, accomplishing peripheral circulation to be evacuated out of the body (Bjorkhem et al., 1998), thus maintaining cholesterol homeostasis. A cDNA sequence for CYP46A1 is disclosed in Genbank Access Number AF094480 (SEQ ID NO:1). The amino acid sequence is shown in SEQ ID NO:2.

The invention makes use of a nucleic acid construct comprising sequence SEQ ID NO:1 or a variant thereof for the treatment of a Polyglutamine repeat spinocerebellar ataxia.

The variants include, for instance, naturally-occurring variants due to allelic variations between individuals (e.g., polymorphisms), alternative splicing forms, etc. The term variant also includes CYP46A1 gene sequences from other sources or organisms. Variants are preferably substantially homologous to SEQ ID NO:1, i.e., exhibit a nucleotide sequence identity of typically at least about 75%, preferably at least about 85%, more preferably at least about 90%, more preferably at least about 95% with SEQ ID NO:1. Variants of a CYP46A1 gene also include nucleic acid sequences, which hybridize to a sequence as defined above (or a complementary strand thereof) under stringent hybridization conditions. Typical stringent hybridisation conditions include temperatures above 30° C., preferably above 35° C., more preferably in excess of 42° C., and/or salinity of less than about 500 mM, preferably less than 200 mM. Hybridization conditions may be adjusted by the skilled person by modifying the temperature, salinity and/or the concentration of other reagents such as SDS, SSC, etc.

### Non-Viral Vectors

In an embodiment, the vector use according to the present invention is a non-viral vector. Typically, the non-viral vector may be a plasmid encoding CYP46A1. This plasmid can be administered directly or through a liposome, an exosome or a nanoparticle.

### Viral Vectors

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins which mediate cell transduction.

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e. g., episomes), or integration of transferred genetic material into the genomic DNA of host cells.

Examples of viral vector include adenovirus, lentivirus, retrovirus, herpes-virus and Adeno-Associated virus (AAV) vectors.

Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478.

In a preferred embodiment, adeno-associated viral (AAV) vectors are employed.

By an “AAV vector” is meant a vector derived from an adeno-associated virus serotype, including without limitation, AAV1, AAV2, AAV3, AAV4, AAV5, AAV6, AAV7, AAV8, AAV9, AAV10, such as AAVrh.10, AAV-PHP.B (Benjamin E. Deverman et al., 2016), etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e. g., functional ITRs) of the virus. The ITRs need not be the wild-type nucleotide sequences, and may be altered, e. g, by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest (i.e. the CYP46A1 gene) and a transcriptional termination region. Two copies of the DNA of interest can be included, as a self-complementary construct (McCarty et al., 2001).

In a more preferred embodiment, the AAV vector is an AAV9 or AAV10 (AAVrh.10) vector, or vector derived from one of these serotypes. In a most preferred embodiment, the AAV vector is an AAVrh.10 vector.

The control elements are selected to be functional in a mammalian cell. The resulting construct which contains the operatively linked components is bounded (5′ and 3′) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome. The nucleotide sequences of AAV ITR regions are known. (See, e. g., Kotin, 1994; Berns, K I “Parvoviridae and their Replication” in Fundamental Virology, 2nd Edition, (B. N. Fields and D. M. Knipe, eds.) for the AAV-2 sequence. As used herein, an “AAV ITR” does not necessarily comprise the wild-type nucleotide sequence, but may be altered, e. g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, etc. Furthermore, 5′ and 3′ ITRs which flank a selected nucleotide sequence in an AAV vector need not necessarily be identical or derived from the same AAV serotype or isolate, so long as they function as intended, i. e., to allow for excision and rescue of the sequence of interest from a host cell genome or vector, and to allow integration of the heterologous sequence into the recipient cell genome when AAV Rep gene products are present in the cell.

Particularly preferred are vectors derived from AAV serotypes having tropism for and high transduction efficiencies in cells of the mammalian central nervous system (CNS), particularly neurons. A review and comparison of transduction efficiencies of different serotypes is provided in Davidson et al., 2000. In one preferred example, AAV2 based vectors have been shown to direct long-term expression of transgenes in CNS, preferably transducing neurons. In other non-limiting examples, preferred vectors include vectors derived from AAV4 and AAV5 serotypes, which have also been shown to transduce cells of the CNS (Davidson et al, supra). In particular, the vector may be an AAV vector comprising a genome derived from AAV5 (in particular the ITRs are AAV5 ITRs) and a capsid derived from AAV5. In another non-limiting example, preferred vectors include vectors obtained by use of cre-recombination-based AAV targeted evolution (CREATE) like AAV-PHP.B.

In a particular embodiment of the invention, the vector is a pseudotyped AAV vector. Specifically, a pseudotyped AAV vector comprises an AAV genome derived from a first AAV serotype and a capsid derived from a second AAV serotype. Preferably, the genome of the AAV vector is derived from AAV2. Furthermore, the capsid is preferably derived from AAV5. Specific non-limiting examples of pseudotyped AAV vectors include an AAV vector comprising a genome derived from AAV2 in a capsid derived from AAV5, an AAV vector comprising a genome derived from AAV2 in a capsid derived from AAVrh.10, etc.

The selected nucleotide sequence is operably linked to control elements that direct the transcription or expression thereof in the subject in vivo. Such control elements can comprise control sequences normally associated with the selected gene. In particular, such control elements may include the promoter of the CYP46A1 gene, in particular the promoter of the human CYP46A1 gene (Ohyama Y et al., 2006)

Alternatively, heterologous control sequences can be employed. Useful heterologous control sequences generally include those derived from sequences encoding mammalian or viral genes. Examples include, but are not limited to, the phosphoglycerate kinase (PGK) promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e. g., Stratagene (San Diego, CA). For purposes of the present invention, both heterologous promoters and other control elements, such as CNS-specific and inducible promoters, enhancers and the like, will be of particular use.

Examples of heterologous promoters include the CMV promoter. Examples of CNS specific promoters include those isolated from the genes from myelin basic protein (MBP), glial fibrillary acid protein (GFAP), synapsins (e.g. human synapsin 1 gene promoter), and neuron specific enolase (NSE).

Examples of inducible promoters include DNA responsive elements for ecdysone, tetracycline, hypoxia andaufin.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e. g., U.S. Pat. Nos. 5,173,414 and 5,139,941; International Publications Nos. WO 92/01070 (published 23 Jan. 1992) and WO 93/03769 (published 4 Mar. 1993); Lebkowski et al., 1988; Vincent et al., 1990; Carter, 1992; Muzyczka, 1992; Kotin, 1994; Shelling and Smith, 1994; and Zhou et al., 1994. Alternatively, AAV ITRs can be excised from the viral genome or from an AAV vector containing the same and fused 5′ and 3′ of a selected nucleic acid construct that is present in another vector using standard ligation techniques. AAV vectors which contain ITRs have been described in, e. g., U.S. Pat. No. 5,139,941. In particular, several AAV vectors are described therein which are available from the American Type Culture Collection (“ATCC”) under Accession Numbers 53222, 53223, 53224, 53225 and 53226. Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. (See, e. g., Edge, 1981; Nambair et al., 1984; Jay et al., 1984). In order to produce rAAV virions, an AAV expression vector is introduced into a suitable host cell using known techniques, such as by transfection. A number of transfection techniques are generally known in the art. (See, e. g., Graham et al., 1973; Sambrook et al. (1989) Molecular Cloning, a laboratory manual, Cold Spring Harbor Laboratories, New York, Davis et al. (1986) Basic Methods in Molecular Biology, Elsevier, and Chu et al., 1981). Particularly suitable transfection methods include calcium phosphate co-precipitation (Graham et al., 1973), direct microinjection into cultured cells (Capecchi, 1980), electroporation (Shigekawa et al., 1988), liposome mediated gene transfer (Mannino et al., 1988), lipid-mediated transduction (Felgner et al., 1987), and nucleic acid delivery using high-velocity microprojectiles (Klein et al., 1987).

For instance, a preferred viral vector, such as the AAVrh.10, comprises, in addition to a cholesterol 24-hydroxylase encoding nucleic acid sequence, the backbone of AAV vector with ITR derived from AAV-2, the promoter, such as the mouse PGK (phosphoglycerate kinase) gene or the cytomegalovirus/β-actin hybrid promoter (CAG) consisting of the enhancer from the cytomegalovirus immediate gene, the promoter, splice donor and intron from the chicken β-actin gene, the splice acceptor from rabbit β-globin, or any neuronal promoter such as the promoter of Dopamine-1 receptor or Dopamine-2 receptor with or without the wild-type or mutant form of woodchuck hepatitis virus post-transcriptional regulatory element (WPRE).

### Delivery of the Vectors

A method of treatment of polyglutamine repeat spinocerebellar ataxia is disclosed, which method comprises administering a vector comprising cholesterol 24-hydroxylase encoding nucleic acid to a patient in need thereof. The vector may be delivered directly into the brain of the subject or by intravascular, intravenous, intranasal, intraventricular or intrathecal injection.

In a particular embodiment, it is provided a method for treating a Polyglutamine repeat spinocerebellar ataxia (PolyQ SCA) in a subject, said method comprising:
(a) providing a vector as defined above, which comprises a cholesterol 24-hydroxylase encoding nucleic acid; and
(b) delivering the vector to the brain of the subject, whereby said vector transduces cells in the brain, and whereby cholesterol 24-hydroxylase is expressed by the transduced cells at a therapeutically effective level.

Advantageously, the vector is a viral vector, more advantageously an AAV vector, even advantageously an AAV9 or AAV10 vector, such as AAVrh.10.

In a particular embodiment, the vector is delivered to cerebellum, striatum, brainstem, frontotemporal lobes, and/or visual cortex, spinal cord, preferably at least to cerebellum. In a particular embodiment, the vector is delivered exclusively to cerebellum.

Methods of delivery, or administration, of viral vectors to neurons and/or astrocytes and/or oligodendrocytes and/or microglia include generally any method suitable for delivery vectors to said cells, directly or through hematopoietic cells transduction, such that at least a portion of cells of a selected synaptically connected cell population is transduced. The vector may be delivered to any cells of the central nervous system, cells of the peripheral nervous system, or both. Preferably, the vector is delivered to cells of the brain. Generally, the vector is delivered to the cells of the brain, including for example cells of brainstem (medulla, pons, and midbrain), cerebellum, substantia nigra, striatum (caudate nucleus and putamen), frontotemporal lobes, visual cortex, spinal cord or combinations thereof, or preferably any suitable subpopulation thereof.

To deliver the vector specifically to a particular region and to a particular population of cells of the brain, the vector may be administered by stereotaxic microinjection. For example, patients have the stereotactic frame base fixed in place (screwed into the skull). The brain with stereotactic frame base (MRI compatible with fiducial markings) is imaged using high resolution MRI. The MRI images are then transferred to a computer which runs stereotactic software. A series of coronal, sagittal and axial images are used to determine the target (site of vector injection) and trajectory. The software directly translates the trajectory into 3 dimensional coordinates appropriate for the stereotactic frame. Burr holes are drilled above the entry site and the stereotactic apparatus positioned with the needle implanted at the given depth. The vector is then injected at the target sites, eventually mixed with a contrast agent. Since the vector integrates into the target cells, rather than producing viral particles, the subsequent spread of the vector is minor, and mainly a function of passive diffusion from the site of injection and of course the desired trans-synaptic transport, prior to integration. The degree of diffusion may be controlled by adjusting the ratio of vector to fluid carrier.

Additional routes of administration may also comprise local application of the vector under direct visualization, e. g., superficial cortical application, intranasal application, or other non-stereotactic application.

The target cells of the vectors of the present invention are cells of the brain of a subject afflicted with PolyQ SCA, preferably neural cells. Preferably the subject is a human being, generally an adult but may be a child or an infant.

However the invention encompasses delivering the vector to biological models of the disease. In that case, the biological model may be any mammal at any stage of development at the time of delivery, e. g., embryonic, foetal, infantile, juvenile or adult, preferably it is an adult. Furthermore, the target cells may be essentially from any source, especially nonhuman primates and mammals of the orders Rodenta (mice, rats, rabbit, hamsters), Carnivora (cats, dogs), and Arteriodactyla (cows, pigs, sheep, goats, horses) as well as any other non-human system (e. g. zebrafish model system).

Preferably, the method of the invention comprises intracerebral administration, through stereotaxic injections. However, other known delivery methods may also be adapted in accordance with the invention. For example, for a more widespread distribution of the vector across the brain, it may be injected into the cerebrospinal fluid, e. g., by lumbar puncture, cisterna magna or ventricular puncture. To direct the vector to the brain, it may be injected into the spinal cord or into the peripheral ganglia, or the flesh (subcutaneously or intramuscularly) of the body part of interest. In certain situations the vector can be administered via an intravascular approach. For example, the vector can be administered intra-arterially (carotid) in situations where the blood-brain barrier is disturbed. Moreover, for more global delivery, the vector can be administered during the “opening” of the blood-brain barrier achieved by infusion of hypertonic solutions including mannitol or ultra-sound local delivery.

The vectors used herein may be formulated in any suitable vehicle for delivery. For instance they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Intravenous vehicles include fluid and nutrient replenishers, electrolyte replenishers (such as those based on Ringer's dextrose), and the like.

Preservatives and other additives may also be present such as, for example, antimicrobials, antioxidants, chelating agents, and inert gases and the like.

A colloidal dispersion system may also be used for targeted gene delivery. Colloidal dispersion systems include macromolecule complexes, nanocapsules, microspheres, beads, and lipid-based systems including oil-in-water emulsions, micelles, mixed micelles, and liposomes or exosomes.

The preferred doses and regimen may be determined by a physician, and depend on the age, sex, weight, of the subject, and the stage of the disease. As an example, for delivery of cholesterol 24-hydroxylase using a viral expression vector, each unit dosage of cholesterol 24-hydroxylase expressing vector may comprise 2.5 to 100 μl of a composition including a viral expression vector in a pharmaceutically acceptable fluid and which provides from 10¹⁰ up to 10¹⁵ cholesterol 24-hydroxylase expressing viral particles per ml of composition.

### Pharmaceutical Composition

A further object of the invention concerns a pharmaceutical composition for use in the treatment of PolyQ SCAs, which comprises a therapeutically effective amount of a vector according to the invention.

By a “therapeutically effective amount” is meant a sufficient amount of the vector of the invention to treat PolyQ SCAs at a reasonable benefit/risk ratio applicable to any medical treatment.

It will be understood that the total daily dosage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range per adult per day. The therapeutically effective amount of the vector according to the invention that should be administered, as well as the dosage for the treatment of a pathological condition with the number of viral or non-viral particles and/or pharmaceutical compositions of the invention, will depend on numerous factors, including the age and condition of the patient, the severity of the disturbance or disorder, the method and frequency of administration and the particular peptide to be used.

The presentation of the pharmaceutical compositions that contain the vector according to the invention may be in any form that is suitable for intramuscular, intracerebral, intranasal, intrathecal, intraventricular or intravenous administration. In the pharmaceutical compositions of the present invention for intramuscular, intranasal, intravenous, intracerebral, intrathecal or intraventricular administration, the active principle, alone or in combination with another active principle, can be administered in a unit administration form, as a mixture with conventional pharmaceutical supports, to animals and human beings.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of sterile injectable solutions.

Upon formulation, solutions will be administered in a manner compatible with the dosage formulation and in such amount as is therapeutically effective. The formulations are easily administered in a variety of dosage forms, such as the type of injectable solutions, but drug release capsules and the like can also be employed.

Multiple doses can also be administered.

The invention will be further illustrated by the following example. However, this example and the accompanying figures should not be interpreted in any way as limiting the scope of the present invention.

### EXAMPLE

The therapeutic benefits of CYP46A1 overexpression was evaluated in two mouse models of PolyQ spinocerebellar ataxia: a lentiviral-based animal model of MJD/SCA3 and a transgenic mouse model of PolyQ with severe established pathology.

### Materials and Methods

### Animals

Two-months aged C57Bl/6 mice (n=8) were obtained by Charles River (Spain) and three-month old transgenic MJD Q69 mice (n=6) and age-matched wild-type littermates (n=6) (Oue et al., 2009; Torashima et al., 2008) were used. MJD transgenic mice (C57BL/6 background) overexpress the N-terminal-truncated human ATXN3, containing 69 CAG repeats and an N-terminal haemagglutinin (HA) tag epitope, driven specifically in cerebellar Purkinje cells by the L7 promoter (Oue et al., 2009). This truncated ATXN3 fragment lacks N-terminal 286 amino-acids that contain the ubiquitin-proteasome domain (Josephin domain) and ubiquitin interaction motifs (UIMs). Therefore, the truncated protein consists of a 69 polyQ tract with only 4 and 42 amino-acids at its N-terminus and C-terminus, respectively. A colony of these transgenic mice was established at the Centre for Neuroscience and Cell Biology of Coimbra. Mice were housed in a temperature-controlled room and maintained on a 12 h light/dark cycle. Food and water were available ad libitum. The experiments were carried out in accordance with the European Community Council directive (2010/63/EU) for the care and use of laboratory animals.

### Lentiviral Vectors Production

Lentiviral vectors encoding human mutant ataxin-3 (LV-PGK-Atx-3 72Q) were produced in HEK293T cell line with a four-plasmid system, as previously described (Alves et al., 2008b). Lentiviral particles were re-suspended in 1% BSA in sterile PBS. The viral particle content of batches was evaluated by assessing HIV-1 p24 antigen levels by ELISA (Retro Tek, Gentaur, Paris, France). Concentrated viral stocks were stored at −80° C. until use.

### AAV Plasmid Design and Vector Production

AAV vectors were produced and purified by Atlantic Gene therapies (INSERM U1089, Nantes, France). Vector production has been described elsewhere (Hudry et al., 2010). The viral constructs for AAVrh.10-GFP and AAVrh.10-CYP46A1 contained the expression cassette consisting of either the Gfp or the human Cyp46a1 genes, driven by a CMV early enhancer/chicken β-actin (CAG) synthetic promoter (CAG) surrounded by inverted terminal repeats (ITR) sequences of AAV2. The AAVrh.10-CAG-GFP vector was also generated and was used as control.

### Stereotactical Injections in the Mouse's Brain

Five-week-old mice were anaesthetized with avertin (14 ml g/1 and 250 mg kg/1 intraperitoneally). Four each animal, 400,000 ng of p24 antigen of lentiviral vectors encoding ATXN3 72Q (MUT ATXN3) and 3.10¹² vg/mL of AAVrh.10 encoding CYP46A1 or the Green Fluorescent protein (GFP; control) in a final volume of 2 μl were stereotactically co-injected (0.2 μl/min) into the striatum in the following coordinates: anteroposterior: +0.6 mm, lateral: ±1.8 mm, ventral: −3.3 mm and tooth bar: 0. After injection, the syringe needle was left in place for an additional 5 min before being slowly raised. The skin was closed using a 6-0 Prolene® suture (Ethicon, Johnson and Johnson, Brussels, Belgium). For cerebellar injections, 6 week-old transgenic MJD Q69 mice (n=25) and age-matched wild-type littermates (n=10) were injected into the cerebellar vermis with AAVrh10-CYP46A1 or AAVrh10-GFP (2 μl; 4.10⁹ vg) at 0.25 ul/min at the coordinates: −1.6 mm rostral to lambda, 0 mm midline, and 1 mm ventral to the skull surface, with the mouth bar set at −3.3.

### Brain Samples

Transgenic MJD Q69 mice (n=6) and age-matched wild-type littermates (n=6) were sacrificed at three months of age. The animals, given an overdose of sodium pentobarbital, were perfused transcardially with ice-cold PBS 0.1M before brain extraction. For western-blot processing, the cerebellum and the “rest of the brain” of the left cerebral hemisphere was dissected and then homogenized in a lysis buffer (TBS, NaCl 150 mM and Triton 1%) containing phosphatase (Pierce) and protease (Roche) inhibitors. After centrifugation (20 min, 13 000 rpm, 4° C.), the supernatant was collected and the protein concentration was quantified (BCA Protein Assay, Thermo Fisher Scientific, Waltham, USA). Lysate aliquots (3 mg of protein/ml) were stored at −80° C. until use. The cerebellum and the “rest of the brain” of the left cerebral hemisphere was dissected and conserved at −80° C. until GC-MS analysis. In addition, C57Bl/6 mice (n=8) injected with viral vectors were perfused transcardially with ice-cold PBS 0.1M, followed by 4% paraformaldehyde (PFA) in 0.1M PBS, before brain extraction. Brains were post-fixed in 4% PFA in 0.1M PBS for 3 days and cryoprotected by incubation in a 20% sucrose/0.1 M PBS solution. Coronal brain sections (25 μm) were cut on a freezing microtome (Leica, Wetzlar, Germany), collected serially, and stored at 4° C., free-floating in 0.02% sodium azide PBS until immunohistochemical processing. The brains of transgenic MJD Q69 mice injected with AAVrh10-CYP46A1 or AAVrh10-GFP and age-matched wild-type mice were removed, post-fixed in 4% PFA for 24 h and cryoprotected by incubation in 25% sucrose/phosphate buffer for 48 h. Subsequently, brains were frozen and sagittal sectioned (40 μm sections) using a cryostat (LEICA CM3050 S).

### Behavioral Tests in Transgenic MJD Mice

To evaluate the impact of AAVrh10-CYP46A1 overexpression on the motor phenotype, transgenic MJD mice (C57BL/6 background) were injected with AAVrh10-CYP46A1 (n=10) or either AAVrh10-GFP (control) (n=9); in addition, four littermate wild-type animals were used. Mice were trained on a battery of motor tests, starting at 5-6 weeks of age, and performed every 4 weeks, starting from week 2 after injection until 10 weeks post-injection. Experiments were assessed by an experienced operator in a blind-fashion manner. All tests were performed in the same dark room after 30 min of acclimatization.

### Rotarod

Motor coordination and balance were evaluated with a Rotarod apparatus (Letica Scientific Instruments). Mice were placed on the Rotarod apparatus (Letica Scientific Instruments), at a constant speed (5 rpm) for a maximum of 5 min and/or at accelerated speed (4-40 rpm, in 5 min) and the latency to fall was recorded. All tests were performed in the same dark room after 30 min of acclimatization. Mice performed four trials for each time point with a 15-20 min rest between trials. For analysis, the mean latency to fall off the Rotarod of three trials was used. Mice were trained and performed this test every 2/4 weeks until 10 weeks after the beginning of the treatment. An experienced operator performed the tests in a blind-fashion manner.

### Footprint Pattern Analysis

The footprint pattern (walking tracks) test was used to compare the gait of mice injected with AAVrh10-CYP46A1 with mice injected with AAVrh10-GFP. Thus, hind and fore feet were coated with red and blue non-toxic paints, respectively. Animals were placed in the floor of a runway (with 15-cm high walls) and walked along a 100-cm long and 10-cm wide sheet of white paper. The footprint patterns were analyzed for the distance between sequential footprints of hind limbs, measured orthogonal to the walking direction. The base between the center of the hind footprint and the center of the preceding hind footprint (hind base) was recorded over a sequence of five consecutive steps, excluding footprints made at the beginning and end of the run.

### Cholesterol and Oxysterol Measurements

Cholesterol and oxysterol analysis followed the ‘gold standard’ method (Dzeletovic et al., 1995) to minimize the formation of autoxidation artefacts. Briefly, mouse striatal tissue samples were weighed and homogenized with a TissueLyser II apparatus (Qiagen) in a 500 μl solution containing butylated hydroxytoluene (BHT, 50 μg/ml) and EDTA (0.5 M). At this point, a mix of internal standards was added [epicoprostanol, ²H₇-7-lathosterol, ²H₆-desmosterol, ²H₆-lanosterol and ²H₇-24(R/S)-hydroxycholesterol] (Avanti Polar Lipids). Alkaline hydrolysis was performed under Ar using 0.35 M ethanolic KOH for 2 h at room temperature. After neutralization of the solution with phosphoric acid, sterols were extracted in chloroform. The lower phase was collected, dried under a stream of nitrogen and the residue was dissolved in toluene. Oxysterols were then separated from the cholesterol and its precursors on a 100 mg Isolute silica cartridge (Biotage); cholesterol was eluted in 0.5% propan-2-ol in hexane followed by oxysterols in 30% propan-2-ol in hexane. The sterol and oxysterol fractions were independently silylated with Regisil®+10% TMCS [bis(trimethylsilyl) trifluoro-acetamide+10% trimethylchlorosilane] (Regis technologies) as described previously (Chevy et al., 2005). The trimethylsilylether derivatives of sterols and oxysterols were separated by gas chromatography (Hewlett-Packard 6890 series) in a medium polarity capillary column RTX-65 (65% diphenyl 35% dimethyl polysiloxane, length 30 m, diameter 0.32 mm, film thickness 0.25 μm; Restesk). The mass spectrometer (Agilent 5975 inert XL) in series with the gas chromatography was set up for detection of positive ions. Ions were produced in the electron impact mode at 70 eV. They were identified by the fragmentogram in the scanning mode and quantified by selective monitoring of the specific ions after normalization and calibration with the appropriate internal and external standards [epicoprostanol m/z 370, ²H₇-7-lathosterol m/z 465, ²H₆-desmosterol m/z 358, ²H₆-lanosterol m/z 504, ²H₇-24(R/S)-hydroxycholesterol m/z 553, cholesterol m/z 329, 7-lathosterol m/z, 7-dehydrocholesterol m/z 325, 8-dehydrocholesterol m/z 325, desmosterol m/z 343, lanosterol m/z 393 and 24(R/S)-hydroxycholesterol m/z 413].

### Western Blot

Total protein concentrations were determined using the BCA kit (Pierce). Equal amounts of total protein extract (30 μg) were electrophoretically separated using SDS-PAGE in 4-12% Bis-Tris gels (NuPAGE® Novex Bis-tris midi gel 15 or 26 wells, Life Technologies, Carlsbad, USA) and transferred to nitrocellulose membranes. Blocked membranes (5% non-fat dry milk in TBS-0.1% Tween-20) were incubated with primary antibodies overnight at 4° C., and washed three times with TBS-0.1% Tween-20 (T-BST) for 10 min. Membranes were then labeled with secondary IgG-HRP antibodies raised against each corresponding primary antibody. After three washes with T-BST, the membranes were incubated with ECL chemiluminescent reagent (Clarity Western ECL substrate; GE Healthcare, Little Chalfont, UK) according to the instructions of the supplier. Peroxydase activity was detected with camera system Fusion TX7 (Fisher Scientific). Normalization was done by densitometry analysis with the Quantity One 1D image analysis software (version 4.4; Biorad, Hercules, CA, USA). The optical densities were normalized with respect to a “standard protein” (GAPDH). A partition ratio was calculated and normalized with respect to the sample with the highest value defined as 1.

### Primary Antibodies

Antibodies used in western-blot (WB) and immunohistochemical (IHC) analyses are listed in table 1, below.

TABLE 1
Antibodies used in western-blot (WB) and immunohistochemical
(IHC) analyses

| Primary antibodies | Source | WB | IHC |
|---|---|---|---|
| mouse anti-ATXN3 (clone 1H9) | Millipore | — | 1:2000 |
| mouse anti hemagglutinin tag | Covance | 1:4000 | 1:5000 |
| rabbit anti-ubiquitin | Dako | — | 1:3000 |
| rabbit anti-CYP46A1 | Abcam | 1:2000 | 1:3000 |
| rabbit anti-DARPP32 | Millipore | — | 1:2000 |
| rabbit anti-GFP | Abcam | — | 1:5000 |
| mouse anti-GAPDH | Abcam | 1:4000 | — |
| rabbit anti-LC3B | Cell Signalling | 1:1000 | — |
| mouse anti-tubulin | Sigma | 1:5000 | — |

### Immunostaining

The immunohistochemical procedure was initiated, by quenching endogenous peroxidase by incubating free-floating sections in phenylhydrazine for 25 min at 37° C. After three washes, slices were blocked in PBS/0.1% Triton X-100 containing 10% Normal Goat Serum (NGS, Gibco) for 1 h at RT. The sections were then incubated with the respective primary antibodies, overnight at 4° C. After three washings, the sections were incubated with the corresponding biotinylated secondary antibody (1:250; Vector Laboratories Inc., CA, USA) diluted in PBS/0.1% Triton X-100 and 10% NGS for 2 h at RT. After three washes, bound antibodies were visualized by the ABC amplification system (Vectastain ABC kit, Vector Laboratories, West Grove, USA) and 3,3′-diaminobenzidine tetrahydrochloride (peroxidase substrate kit, DAB, Vector Laboratories, CA, USA) as the substrate. The sections were mounted, dehydrated by passing twice through ethanol and toluol solutions, and coverslipped with Eukitt® (O. Kindler GmbH & CO, Freiburg, Germany).

### Image Acquisition

Images of immunostained sections were acquired with a Z6 APO macroscope (Leica) and LAS V3.8 (Leica) software, at room temperature, with a brightfield Leica DM 5000B microscope equipped with a Leica DFC310FX digital camera. Photographs for comparison were taken under identical conditions of image acquisition, and all adjustments of brightness and contrast were applied uniformly to all images.

### Immunohistochemical Quantitative Analysis of ATXN3 Aggregates and DARPP-32 Depletion in the LV-Based Model of MJD

Brain slices were analyzed and imaged at RT with an Axiovert 200M microscope (CCD color digital Axiocam HRc camera; Carl Zeiss), at 5× (air objective, Fluar, 0.25 NA) and 20× magnifications (air objective, LD-PlanNeofluar, 0.4 NA). Composite images of complete aggregate-containing or DARPP-32-depleted regions were automatically acquired using the MozaiX function of the AxioVision software. Quantification of ATXN3-positive aggregates and determination of the area of the DARPP-32 depletion was made in 7-8 slices for each mouse, spread over the anterior-posterior extent of the striatum and separated by 200 μm. Counting of ATXN3-positive aggregates was performed on 20×-magnified images of each hemisphere using ImageJ (National Institutes of Health). After manually thresholding images so that the recognizable ATXN3-positive accumulations were included in the examination, particles>3 μm² were automatically analyzed and counted (scale: 2 pixels/μm), and the estimative calculation of the total number of aggregates in the entire striatum was performed as previously described (Alves et al., 2008a; Alves et al., 2010). The extent of MUT ATXN3 lesions in the striatum was analyzed by digitizing 6-8 DARPP-32-stained sections per animal (200 μm between 25 μm thickness sections), selected to obtain a complete rostro-caudal sampling of the striatum with a slide scanner and by quantifying the area of the lesion with a semiautomated image-analysis program (ImageJ software, NIH, MD, USA), to manually define the regions with decreased antibody reactivity and quantify their area (scale: 0.5 pixels/μm). Sections throughout the entire striatum were analyzed. The area of the striatum showing a loss of DARPP-32 staining was measured for each animal with an operator-independent macro. The volume was then estimated using the following formula: volume=d(a1+a2+a3+ . . . ), where d is the distance between serial sections (200 μm), and a1, a2, a3 etc. are DARPP-32-depleted areas for individual serial sections (Alves et al., 2008b). The average gray value of all pixels measured in the lesioned area was recorded for each depleted area. Results are presented as the calculated value for volume of the DARPP-32 depleted region for each animal. In all cases, imaging, measurements, and quantifications were performed blindly to the treatment.

### Immunohistochemistry Quantitative Analysis in the MJD Transgenic Mice

Quantification of cerebellar volume: The cerebellar volume was blindly assessed by scanning the area per lobe in 8 sections stained with cresyl violet, covering the full extent of the cerebellum. The cerebellar volume was obtained by multiplying the total area by the distance between serial sections (320 μm).

Quantification of granular and molecular layers size: Quantification was made over 4 cresyl violet staining sections spread over the anterior-posterior extent of the cerebellum in a blind fashion using a 20× objective. For each section the size of the layers was assessed blindly per lobe in 3 to 5 different measurements (depending on the lobe size).

Quantification of Purkinje cells: Fluorescence images of 8 sagittal sections covering entire cerebellum, stained with anti-Calbindin and DAPI were acquired with a Zeiss Axio Imager Z2 microscope using a 20× objective. For each section and the number of Purkinje cells were manually counted for each cerebellar lobe in a blind fashion way. Data is represented as mean value of the number of Purkinje cells in the entire cerebellum or per lobe.

Quantification of ubiquitin aggregates: The number of ubiquitin aggregates was assessed in lobes 9 and 10 automatically using Cell Profiler software, from images 8 sagittal sections covering entire cerebellum, stained with anti-ubiquitin and DAPI were acquired with a Zeiss Axio Imager Z2 microscope using a 20× objective. The analysis modules applied were the same for all the animals and images. Data is represented as the average number of ubiquitin aggregates per section.

### Cresyl Violet Staining

Cerebellar sections were stained with cresyl violet for 2 minutes, differentiated in acetate buffer pH 3.8 to 4 (2.72% sodium acetate and 1.2% acetic acid; 1:4 v/v), dehydrated by passing twice through ethanol and toluol solutions, and mounted with Eukitt® (O. Kindler GmbH & CO. Freiburg, Germany).

### Neuroblastoma Cell Culture and Transfection

Mouse neuroblastoma cell line (N2a cells) obtained from the American Type Culture Collection cell biology bank (CCL-131) were maintained in Dulbecco's modified Eagle's medium (DMEM) supplemented with 10% foetal bovine serum (FBS), 100 U/ml penicillin and 100 mg/ml streptomycin (Gibco) at 37° C. in 5% CO₂/air atmosphere. The human full-length mutant Atx3 with 84 glutamines (pEGFP-Atx3Q84; (Chai et al., 2002) was transfected in the Neuro2A cells using PEI (Polyethylenimine) and a total concentration of 0.5 mg of each DNA. Cells were lysed for western blot processing 48 hours post-transfection.

### Autophagic Flux Measurement

The autophagic flux was measured through the LC3B turnover assay, which is used to quantify the amount of LC3B-II that is delivered to the lysosomes (Aveleira et al., 2015). For that purpose, the levels of LC3B-II were measured in the presence and absence of chloroquine (ChQ, SIGMA, 100 μm), an inhibitor of autolysosomal degradation. The autophagic flux was calculated by subtracting the densitometric value of LC3B-II in samples without chloroquine (LC3B-II-ChQ) from the corresponding sample treated with chloroquine (LC3B-II+ChQ) for each condition.

### Statistical Analysis

Statistical analysis was performed using the Mann-Whitney test and paired or unpaired Student's t-test or ANOVA for multiple comparisons. Results are expressed as mean±SEM. Significant thresholds were set at P<0.05, P<0.01 and P<0.001, as defined in the text. All analyses were performed using GraphPad Prism (GraphPad Software, La Jolla, USA).

### Results

### Decreased Levels of CYP46A1 Protein Levels in the Cerebellum of MJD Transgenic Mice

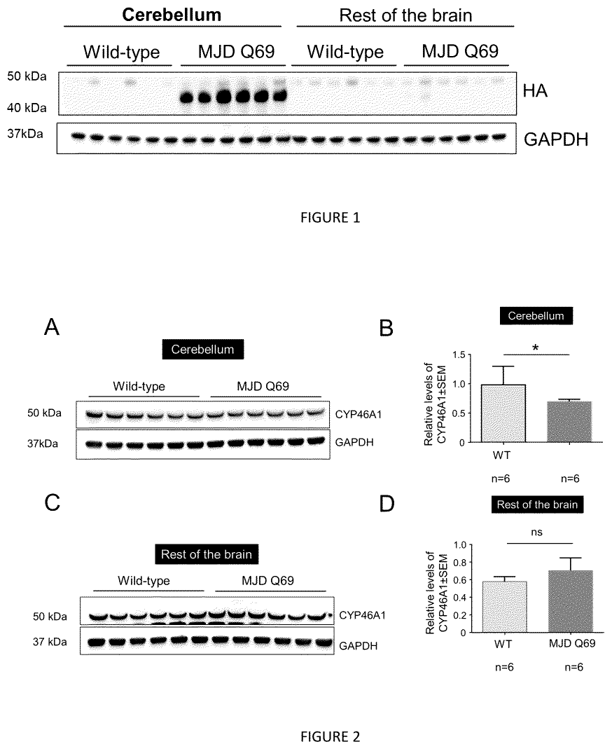

The levels of CYP46A1 was evaluated in frozen biopsies from six MJD transgenic mice expressing the N-terminal truncated human ATXN3, with 69 CAG repeats and six age-matched control littermates by western-blot. In particular, CYP46A1 protein levels was analyzed in the cerebellum, since mutant human ataxin-3 was specifically expressed in cerebellar Purkinje cells (Oue et al., 2009). As control region, the “rest of the brain” (all brain tissue excepting the cerebellum) was used, in which no expression of mutant human ataxin-3 was detected (FIG. 1). Western-blot analysis demonstrated a 24.3% reduction in CYP46A1 protein levels (Mann-Whitney test; p=0.026) in cerebellar biopsies from MJD mice relatively to control littermates (FIGS. 2A and B). In the contrary, no statistical significant differences were found in extracts from the “rest of the brain” among MJD mice and control littermates (Mann-Whitney test; p=0.13) (FIGS. 2C and D).

Overall, these data support the relevance of CYP46A1, a key enzyme in cholesterol metabolism pathway, as a therapeutic target in human spinocerebellar ataxias, in particular, MJD.

### Deregulation of the Cholesterol Metabolism Pathway in the Brain of MJD Transgenic Mice

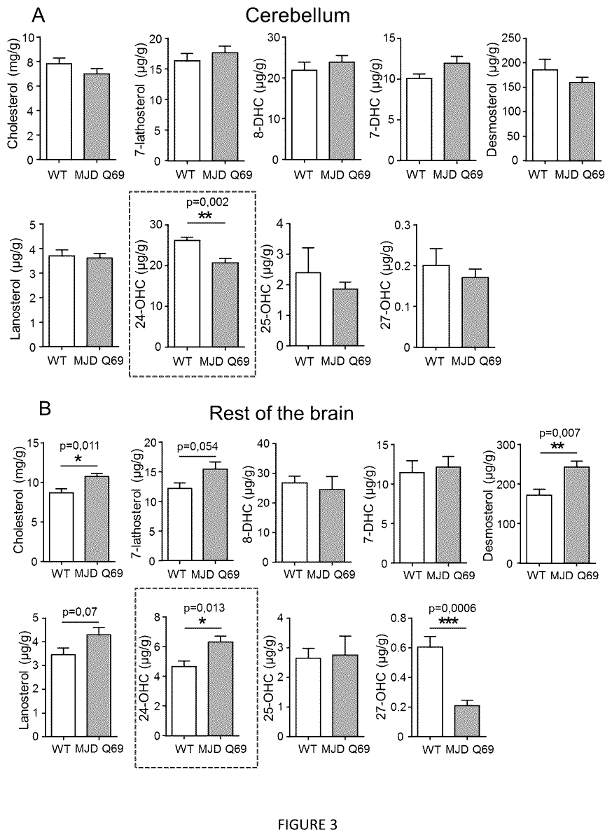

To determine the impact of CYP46A1 deficiency in MJD, the cholesterol metabolism was analyzed by performing quantitative measurements of both sterols and oxysterols by gas-chromatography mass spectrometry (GC/MS) in cerebellar extracts from MJD Q69 mice and age-matched littermates. Therefore, lanosterol, the first sterol of the cholesterol metabolic pathway, along with cholesterol intermediates from the Kandutsch-Russell (lathosterol) and Bloch (desmosterol) pathways were measured. The cholesterol and 24S-OHC content were first compared and a statistically significant reduction (21.3%) was observed in the levels of 24S-OHC in MJD Q69 mice (20.62±1.14 μg/g; n=6) relatively to wild-type littermates (26.19±0.78 μg/g; n=6, Student's T test, P=0.002) (FIG. 3A). No statistically significant differences were observed for the cerebellar cholesterol content among MJD mice and wild-type mice (FIG. 3A). No statistically significant differences were found for the oxysterols 25S-OHC and 27S-OHC and for the remaining sterols (lanosterol, 7-lathosterol, desmosterol, 7-DHC and 8-DHC) (FIG. 3A).

As mutant human ataxin-3 is only expressed in cerebellar Purkinje cells, the “rest of the brain” was used to measure the levels of the above referred oxysterols and sterols. Surprisingly, a statistically significant increase was detected in the levels of 24S-OHC in the “rest of the brain” from MJD Q69 mice (6.32±0.40 μg/g; n=6), relatively to control littermates (4.68±0.37 μg/g; n=6; Student's T test, P=0.013) (FIG. 3B). A statistically significant augmentation was also found in the levels of cholesterol in MJD Q69 mice (10.74±0.41 mg/g; n=6) compared to wild-type mice (8.68±0.53 mg/g; n=6; Student's T test, P=0.011) (FIG. 3B). The levels of the cholesterol precursor desmosterol were increased in MJD Q69 mice (243.0±14.93 μg/g; n=6) relatively to wild-type mice (172.5±14.6 μg/g; n=6; Student's T test, P=0.007), as well as, the levels of 7-lathosterol (4.68±0.37 μg/g in normal mice; n=6 vs 6.32±0.40 μg/g in MJD Q69 mice; n=6; Student's T test, P=0.054) (FIG. 3B). In the same line, the levels of lanosterol were slightly increased in MJD Q69 mice (4.30±0.30 μg/g; n=6) as compared to wild-type mice (3.46±0.29 μg/g; n=6, Student's T test, P=0.07) (FIG. 3B). No statistical significant differences were found for the 7-dehydrocholesterol (7-DHC) and for the 8-dehydrocholesterol (8-DHC). In addition, the levels of the oxysterol 27-OHC were decreased in MJD Q69 mice (0.21±0.035 μg/g; n=6) relatively to wild-type mice (0.60±0.07 μg/g; n=6; Student's T test P=0.0006). No differences were observed for 25-OHC levels among MJD Q69 and wild-type mice (FIG. 3B).

Altogether, these results suggest alterations in the cholesterol metabolism pathway in MJD affected regions like the cerebellum (in which mutant ATXN3 is specifically expressed) and also in regions in which no mutant ATXN3 is detected (rest of the brain).

### CYP46A1 Overexpression Alleviates Neuropathology in an MJD LV-Based Mouse Model

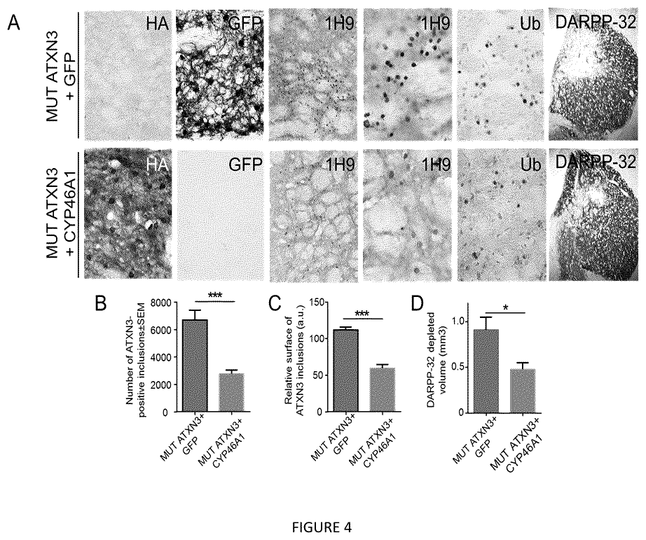

The invention then investigated whether the upregulation of the cholesterol metabolism pathway, through increase in the levels of CYP46A1, could improve mutant ataxin-3 clearance, therefore delaying the establishment of MJD-associated neuropathology. Next, the therapeutic effect of CYP46A1 overexpression in the LV-based mouse model of MJD was evaluated (Alves et al., 2008b; Nobrega et al., 2013). For this purpose, LV encoding mutant human ataxin-3 and an AAV encoding CYP46A1 were co-expressed in the striatum of an MJD mouse model and sacrificed the mice two months post-injection. HA-directed immunohistochemistry revealed the presence of the HA tag in AAVrh.10-CYP46A1-injected LV-MJD mice but not in the control group injected with AAVrh.10-GFP (FIG. 4A). Conversely, in mice injected with AAVrh.10-GFP, a robust and widespread GFP immunoreactivity was detected in the mouse brain, whereas no HA-positive signals were detected (FIG. 4A).

In the MJD mouse model, immunohistochemistry (with the 1H9 antibody) and quantitative analysis for mutant ataxin-3-positive inclusions (FIG. 4A) revealed a statistically significant CYP46A1-mediated decrease (˜59%) in the number of ataxin-3-positive inclusions (2751±267.8; n=8) compared to the control group (GFP-co-injected mice; 6684±717.2 inclusions; n=8; Student's T test, P=0.0002) (FIG. 4B). The surface of inclusions was also analyzed and demonstrated a statistically significant decrease in the size of inclusions (˜47%) in AAVrh.10-CYP46A1 treated LV-MJD mice (59.29±4.67 arbitrary units (a.u.); n=8) compared to LV-MJD mice injected with the control AAVrh.10-GFP (111.4±3.96 a.u.; n=8; Student's T test, P<0.0001) (FIG. 4B). These results were completed with anti-ubiquitin immunostaining that demonstrated a reduction in the number of ubiquitinated inclusions in LV-MJD mice injected with AAVrh.10-CYP46A1 relatively to LV-MJD mice in which the control AAVrh.10-GFP was injected (FIG. 4A). In view of these results, whether mutant human ataxin-3 clearance would confer neuroprotection in the LV-MJD mouse model was next inspected. It has been reported that LV-based mutant ataxin-3 overexpression in the mouse striatum promotes depletion of the neuronal marker DARPP-32, that can be precisely quantified (Alves et al., 2008b). Therefore immunohistochemistry for DARPP-32 was performed (FIG. 4D). CYP46A1 overexpression led to a statistically significant reduction (˜48%) of neuronal dysfunction, as assessed by decreased DARPP-32 depleted region at 2 months (0.48±0.77 mm³; n=8) relatively to LV-MJD mice injected with control AAVrh.10-GFP (0.91=0.14 mm³; n=8) Student's T test; P=0.015).

Overall, these data support that CYP46A1 overexpression has therapeutic properties, promoting the clearance of misfolded mutant human ataxin-3, therefore conferring preservation of neuronal markers and neuroprotection in an LV-MJD mouse model.

### AAV-Mediated CYP46A1 Gene Therapy Improves Neuropathology and Motor Coordination in MJD Transgenic Mice

Next investigation if CYP46A1 upregulation would alleviate motor and cerebellar deficits after the onset of the disease was performed, by using a transgenic MJD mouse model (Torashima et al., 2008). This model is characterized by a strong motor and neuropathological phenotype and particularly suitable for testing molecular therapies (Nobrega et al., 2014; Mendonca et al., 2015; Nobrega et al., 2015). To assess the potential of CYP46A1 overexpression to improve the motor phenotype of MJD-transgenic mice with severe motor impairments, AAVrh10 encoding for CYP46A1, or GFP (control), were injected into the cerebellum of adult MJD-transgenic mice and the motor performance was evaluated every 2/4 weeks until 10 weeks after the virus injection. Using rotarod test at constant speed (FIG. 5A) and accelerated rotarod test (FIG. 5B) it was observed that at the time of the injection the MJD-transgenic mice already displayed marked motor impairments, as compared to wild-type mice (WT). Moreover, throughout the study the mice expressing CYP46A1 have a better motor performance as compared to GFP mice, although, only at 10 weeks after AAV injection the different latency to fall observed between mice expressing CYP46A1 and GFP became significant (FIG. 5A, CYP 53.07 s versus GFP 17.48 s) for both rotarod tests. This improvement in the motor function was further analyzed with foot printing pattern test, namely the hind base test. It was observed that mice treated with AAVrh10-CYP46A1 have a tendency for a better hind base performance at 10 weeks after the virus injection (FIG. 5C, AAVrh10-CYP46A1: 3.17±0.07 cm versus AAVrh10-GFP: 3.40±0.11 cm); however no significant difference was observed between both groups with this test.

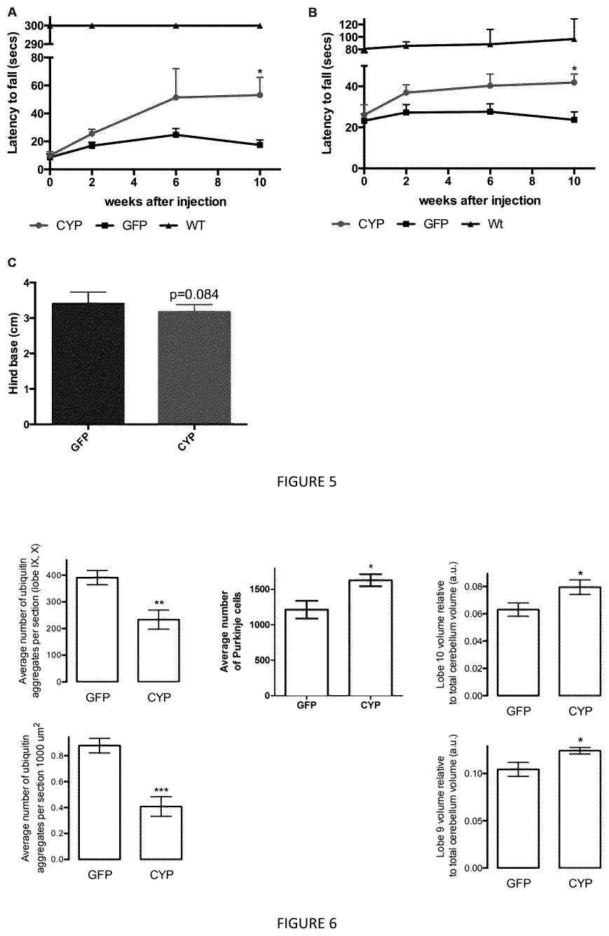

To investigate whether the improvements in motor function were also related with neuronal improvements, the aggregates number and the cerebellar neuropathology of the injected mice were next evaluated. Like in (Nobrega et al., 2013b; Nobrega et al., 2014), the transduction of viral vectors was preferentially detected on lobes IX and X of cerebellar cortex. In accordance with behavioural data, the average number ubiquitin aggregates were significantly reduced in transduced lobes in AAVrh10-CYP46A1-injected mice compared with AAVrh10-GFP-injected controls (FIG. 6). This transgenic mouse model is characterized by a loss of Purkinje cells and a strong disorganization of cerebellar cortex (Oue et al., 2009). Importantly, the average number of Purkinje cells was significantly higher in animals injected with AAVrh10-CYP46A1 compared to the controls (FIG. 6). Moreover, the cerebellar volume was higher in lobe IX and X in AAVrh10-CYP46A1-injected mice, as well as the thickness of granular layer (FIG. 6). Altogether these data show that CYP46A1 upregulation mediates a robust alleviation of motor phenotype and neuropathological abnormalities in a severe-progressive MJD transgenic mouse model.

### CYP46A1 Overexpression Induces Autophagy and Clears Mutant ATXN3 in Neuro2A Cells

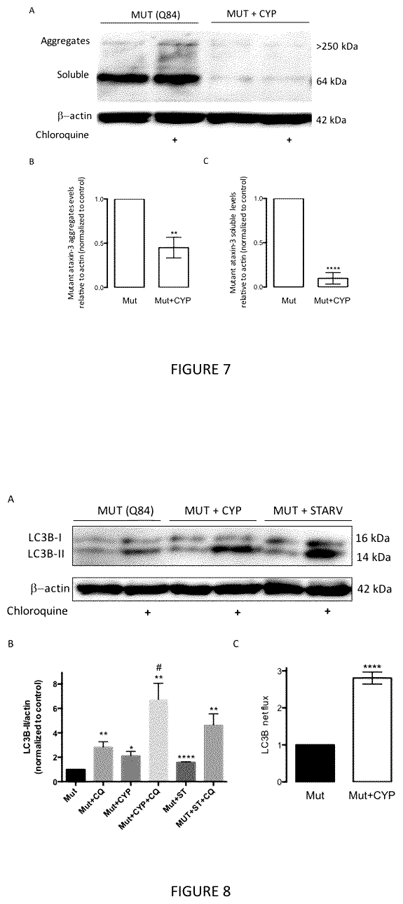

The molecular mechanism of CYP46A1 action and its impact in Neuro2A cells expressing mutant ATXN3 (EGFP-Q84) were next investigated. The analysis of mutant ATXN3 species levels by western blot revealed that CYP46A1 overexpression significantly reduce those levels compared to control conditions (FIG. 7A), both in aggregates (1±0.0 vs 0.449±0.11; n=4; FIG. 7B), as well as in the soluble protein (1±0.0 vs 0.09±0.06; n=4; FIG. 7C). Based on these results, investigations were next performed to determine if CYP46A1 was reducing mutant ATXN3 through autophagy, as a main cellular pathway for clearing aggregated species (FIG. 8A). In fact, it was found that CYP46A1 overexpression significantly increased LC3B-II levels (2.12±0.35; n=4) comparing to the control condition (1±0.0; n=4). This increase in the LC3B-II levels was even higher upon CYP46A1 overexpression compared to the starvation condition (autophagy positive control; 1.58±0.02; n=4) (FIG. 8B). Moreover, in the presence of the autophagy inhibitor (chloroquine—ChQ) the results observed follow the same trend, i.e., an increase in LC3B-II levels upon CYP46A1 overexpression (6.74±1.31; n=4) compared to control condition (2.83±0.44; n=4) (FIG. 8B). Moreover, CYP46A1 overexpression significantly increased the autophagic flux, highlighted by an increase in the LC3B-II net flux (2.80±0.16; n=4) comparing to the control condition (1±0.0; n=4) (FIG. 8C).

Altogether these results show that CYP46A1 is a strong modulator of mutant ATXN3, reducing its levels through a robust activation of the autophagy pathway.

Thus, it was demonstrated that CYP46A1 levels are decreased in cerebellar extracts from MJD/PolyQ mice. It was also showed that overexpression of AAVrh10-CYP46A1 in a lentiviral-based animal mouse model of MJD conferred neuroprotection by reducing mutant human ataxin-3 accumulation, associated to the preservation of neuronal markers. This was confirmed in adult MJD transgenic mice, in which cerebellar AAVrh10-CYP46A1 overexpression induced a noteworthy alleviation of motor behavior impairments correlated with mitigation of MJD-associated neuropathology, in particular, decrease of misfolded protein and reduction of Purkinje cell loss and of cellular layer shrinkage in the cerebellum. Thus, AAV-CYP46A1 reduces the accumulation of misfolded ataxin-3 alleviating MJD-associated neuropathology, therefore improving motor behavior defects.

Remarkably, it was demonstrated in neuroblastoma cells that CYP46A1 activates autophagy decreasing mutant ATXN3 deposition.

Therefore, these results support CYP46A1 as a relevant therapeutic target for SCAs.

### REFERENCES

Alves, S., et al., 2008a. Allele-specific RNA silencing of mutant ataxin-3 mediates neuroprotection in a rat model of Machado-Joseph disease. PLoS One. 3, e3341.

Alves, S., et al., 2008b. Striatal and nigral pathology in a lentiviral rat model of Machado-Joseph disease. Hum Mol Genet. 17, 2071-83.

Alves, S., et al., 2010. Silencing ataxin-3 mitigates degeneration in a rat model of Machado-Joseph disease: no role for wild-type ataxin-3? Hum Mol Genet. 19, 2380-94.

Bjorkhem, I., et al., 1998. Cholesterol homeostasis in human brain: turnover of 24S-hydroxycholesterol and evidence for a cerebral origin of most of this oxysterol in the circulation. J Lipid Res. 39, 1594-600.

Burk, K., et al., 1999. Autosomal dominant cerebellar ataxia type I: oculomotor abnormalities in families with SCA1, SCA2, and SCA3. J Neurol. 246, 789-97.

Chevy, F., Humbert, L., Wolf, C., 2005. Sterol profiling of amniotic fluid: a routine method for the detection of distal cholesterol synthesis deficit. Prenat Diagn. 25, 1000-6.

Durr, A., et al., 1996. Spinocerebellar ataxia 3 and Machado-Joseph disease: Clinical, molecular, and neuropathological features. Annals of Neurology. 39, 490-499.

Dzeletovic, S., et al., 1995. Time course of oxysterol formation during in vitro oxidation of low density lipoprotein. Chem Phys Lipids. 78, 119-28.

Hudry, E., et al., 2010. Adeno-associated virus gene therapy with cholesterol 24-hydroxylase reduces the amyloid pathology before or after the onset of amyloid plaques in mouse models of Alzheimer's disease. Mol Ther. 18, 44-53.

Kawaguchi, Y., et al., 1994. CAG expansions in a novel gene for Machado-Joseph disease at chromosome 14q32.1. Nat Genet. 8, 221-8.

Kotin, et al. 1994. Parvoviridae and their Replication. Fundamental Virology.

Klockgether, T., et al., 1998. Autosomal dominant cerebellar ataxia type I. MRI-based volumetry of posterior fossa structures and basal ganglia in spinocerebellar ataxia types 1, 2 and 3. Brain. 121 (Pt 9), 1687-93.

Lukas C., et al., 2008. Spinal cord atrophy in spinocerebellar ataxia type 3 and 6: impact on clinical disability. J Neurol. 2008 August; 255(8):1244-9. doi: 10.1007/s00415-008-0907-6. Epub 2008 Jun. 5.

McCarty, D M., et al., 2001. Self-complementary recombinant adeno-associated virus (scAAV) vectors promote efficient transduction independently of DNA synthesis. gene Ther. 2001 August; 8(16):1248-54.

Nobrega, C., et al., 2013. Overexpression of mutant ataxin-3 in mouse cerebellum induces ataxia and cerebellar neuropathology. Cerebellum. 12, 441-55.

Orr, H. T., Zoghbi, H. Y., 2007. Trinucleotide repeat disorders. Annu Rev Neurosci. 30, 575-621.

Oue, M., et al., 2009. Characterization of mutant mice that express polyglutamine in cerebellar Purkinje cells. Brain Res. 1255, 9-17.

Rub, U., et al., 2006. Degeneration of ingestion-related brainstem nuclei in spinocerebellar ataxia type 2, 3, 6 and 7. Neuropathol Appl Neurobiol. 32, 635-49.

Rub, U., Brunt, E. R., Deller, T., 2008. New insights into the pathoanatomy of spinocerebellar ataxia type 3 (Machado-Joseph disease). Curr Opin Neurol. 21, 111-6.

Schols, L., et al., 1997. Autosomal dominant cerebellar ataxia: phenotypic differences in genetically defined subtypes? Ann Neurol. 42, 924-32.

Schols, L., et al., 2004. Autosomal dominant cerebellar ataxias: clinical features, genetics, and pathogenesis. Lancet Neurol. 3, 291-304.

Torashima, T., et al., 2008. Lentivector-mediated rescue from cerebellar ataxia in a mouse model of spinocerebellar ataxia. EMBO Rep. 9, 393-9.

Benjamin E. Deverman et al., 2016. Cre-dependent selection yields AAV variants for widespread gene transfer to the adult brain. Nat Biotechnol. 34(2):204-209.

1.-20. (canceled)

21. A vector for treating a Polyglutamine repeat spinocerebellar ataxia, which vector comprises cholesterol 24-hydroxylase encoding nucleic acid.

22. The vector of claim 21, wherein the Polyglutamine repeat spinocerebellar ataxia is selected from the group of Spinocerebellar ataxia type 1 (SCA1), Spinocerebellar ataxia type 2 (SCA2), Spinocerebellar ataxia type 3 (SCA3), Spinocerebellar ataxia type 6 (SCA6). Spinocerebellar ataxia type 7 (SCA7) and Spinocerebellar ataxia type 17 (SCA17).

23. The vector of claim 21, wherein the Polyglutamine repeat spinocerebellar ataxia is Spinocerebellar ataxia type 3 (SCA3).

24. The vector of claim 21, which is selected from the group of adenovirus, lentivirus, retrovirus, herpesvirus and Adeno-Associated Virus (AAV) vectors.

25. The vector of claim 24, which is an AAV vector.

26. The vector of claim 25, which is an AAV9 or AAV10 vector, such as AAVrh.10, preferably an AAVrh.10.

27. The of claim 21, formulated for administration directly into the brain of a patient.

28. The vector of claim 27, formulated for administration to cerebellum, brainstem, substantia nigra, striatum, frontotemporal lobes and/or visual cortex, preferably to cerebellum.

29. The vector of claim 21 formulated for administration into the spinal cord of a patient.

30. A vector for use in the treatment of a Polyglutamine repeat spinocerebellar ataxia, which vector comprises cholesterol 24-hydroxylase encoding nucleic acid.

31. The vector for use in the treatment of a Polyglutamine repeat spinocerebellar ataxia according to claim 30, wherein the Polyglutamine repeat spinocerebellar ataxia is selected from the group of Spinocerebellar ataxia type 1 (SCA1), Spinocerebellar ataxia type 2 (SCA2), Spinocerebellar ataxia type 3 (SCA3), Spinocerebellar ataxia type 6 (SCA6), Spinocerebellar ataxia type 7 (SCA7) and Spinocerebellar ataxia type 17 (SCA17).

32. The vector for use in the treatment of a Polyglutamine repeat spinocerebellar ataxia according to claim 30, wherein the Polyglutamine repeat spinocerebellar ataxia is Spinocerebellar ataxia type 3 (SCA3).

33. (canceled)

34. (canceled)

35. The vector for use in the treatment of a Polyglutamine repeat spinocerebellar ataxia according to claim 30, which is selected from the group of adenovirus, lentivirus, retrovirus, herpesvirus and Adeno-Associated Virus (AAV) vectors.

36. The vector for use in the treatment of a Polyglutamine repeat spinocerebellar ataxia according to any one of claim 35, which is an AAV vector.

37. The vector for use in the treatment of a Polyglutamine repeat spinocerebellar ataxia according to claim 36, which is an AAV9 or AAV10 vector, such as AAVrh.10, preferably an AAVrh.10.

38. The vector for use in the treatment of a Polyglutamine repeat spinocerebellar ataxia according to claim 37, which is to be administered directly into the brain of the patient.

39. The vector for use in the treatment of a Polyglutamine repeat spinocerebellar ataxia according to claim 39, which is to be administered to cerebellum, brainstem, substantia nigra, striatum, frontotemporal lobes and/or visual cortex, preferably to cerebellum.

40. A pharmaceutical composition for use in the treatment of Polyglutamine repeat spinocerebellar ataxias, which comprises a therapeutically effective amount of a vector as defined in claim 30.


SEQUENCE LISTING (2 sequences)

SEQ ID NO: 1 - DNA, 1503 bp, Homo sapiens
  source 1..1503: mol_type = unassigned DNA
  CDS 1..1503
atgagccccgggctgctgctgctcggcagcgccgtcctgctcgccttcggcctctgctgcaccttcgtgcaccgcgctcgcagccgctacgagcacatccccgggccgccgcggcccagtttccttctaggacacctcccctgcttttggaaaaaggatgaggttggtggccgtgtgctccaagatgtgtttttggattgggctaagaagtatggacctgttgtgcgggtcaacgtcttccacaaaacctcagtcatcgtcacgagtcctgagtcggttaagaagttcctgatgtcaaccaagtacaacaaggactccaagatgtaccgtgcgctccagactgtgtttggtgagagactcttcggccaaggcttggtgtccgaatgcaactatgagcgctggcacaagcagcggagagtcatagacctggccttcagccggagctccttggttagcttaatggaaacattcaacgagaaggctgagcagctggtggagattctagaagccaaggcagatgggcagaccccagtgtccatgcaggacatgctgacctacaccgccatggacatcctggccaaggcagcttttgggatggagaccagtatgctgctgggtgcccagaagcctctgtcccaggcagtgaaacttatgttggagggaatcactgcgtcccgcaacactctggcaaagttcctgccagggaagaggaagcagctccgggaggtccgggagagcattcgcttcctgcgccaggtgggcagggactgggtccagcgccgccgggaagccctgaagaggggcgaggaggttcctgccgacatcctcacacagattctgaaagctgaagagggagcccaggacgacgagggtctgctggacaacttcgtcaccttcttcattgctggtcacgagacctctgccaaccacttggcgttcacagtgatggagctgtctcgccagccagagatcgtggcaaggctgcaggccgaggtggatgaggtcattggttctaagaggtacctggatttcgaggacctggggagactgcagtacctgtcccaggtcctcaaagagtcgctgaggctgtacccaccagcatggggcacctttcgcctgctggaagaggagaccttgattgatggggtcagagtccccggcaacaccccgctcttgttcagcacctatgtcatggggcggatggacacatactttgaggacccgctgactttcaaccccgatcgcttcggccctggagcacccaagccacggttcacctacttccccttctccctgggccaccgctcctgcatcgggcagcagtttgctcagatggaggtgaaggtggtcatggcaaagctgctgcagaggctggagttccggctggtgcccgggcagcgcttcgggctgcaggagcaggccacactcaagccactggaccccgtgctgtgcaccctgcggccccgcggctggcagcccgcacccccaccacccccctgctga

SEQ ID NO: 2 - protein, 500 aa, Homo sapiens
MSPGLLLLGSAVLLAFGLCCTFVHRARSRYEHIPGPPRPSFLLGHLPCFWKKDEVGGRVLQDVFLDWAKKYGPVVRVNVFHKTSVIVTSPESVKKFLMSTKYNKDSKMYRALQTVFGERLFGQGLVSECNYERWHKQRRVIDLAFSRSSLVSLMETFNEKAEQLVEILEAKADGQTPVSMQDMLTYTAMDILAKAAFGMETSMLLGAQKPLSQAVKLMLEGITASRNTLAKFLPGKRKQLREVRESIRFLRQVGRDWVQRRREALKRGEEVPADILTQILKAEEGAQDDEGLLDNFVTFFIAGHETSANHLAFTVMELSRQPEIVARLQAEVDEVIGSKRYLDFEDLGRLQYLSQVLKESLRLYPPAWGTFRLLEEETLIDGVRVPGNTPLLFSTYVMGRMDTYFEDPLTFNPDRFGPGAPKPRFTYFPFSLGHRSCIGQQFAQMEVKVVMAKLLQRLEFRLVPGQRFGLQEQATLKPLDPVLCTLRPRGWQPAPPPPPC